(19) United States
(12) Patent Application Publication
(10) Pub. No.: US 2020/0163824 A1
(43) Pub. Date: May 28, 2020

(54) SYSTEM AND METHOD FOR CONTROLLING INDIVIDUALLY CUSTOMIZED SLEEP MASSAGE CHAIR USING DATA OF SLEEP STATE

(71) Applicant: Blaubit Co., Ltd., Seongnam,-si (KR)

(72) Inventor: Euisun KIM, Seoul (KR)

(73) Assignee: Blaubit Co., Ltd., Seongnam-si (KR)

(21) Appl. No.: 16/227,498

(22) Filed: Dec. 20, 2018

(30) Foreign Application Priority Data
Nov. 26, 2018 (KR) 10-2018-0146909

(51) Int. Cl. A61H 15/00; A61B 5/00; G06K 9/00

(52) U.S. Cl. CPC A61H 15/0078; A61B 5/4815; A61H 2201/0149; G06K 9/00302; A61B 5/4809

(57) ABSTRACT

Disclosed are an individually customized sleep massage chair using sleep state data and a control method thereof. The present disclosure induces a user to enter the sleep state, analyzes the sleep state of the user, thereby providing a result thereof, and performs control according to the analysis result such that the shape of the chair is changed so as to maintain the sleep state of the user.

### CROSS-REFERENCE TO RELATED APPLICATION(S)

This application claims the benefit under 35 USC 119(a) of Korean Patent Application No. 10-2018-0146909 filed on Nov. 26, 2018, in the Korean Intellectual Property Office, the entire disclosure of which is incorporated herein by reference for all purposes.

### BACKGROUND OF THE INVENTION

### 1. Field of the Invention

The present disclosure relate to a system and a method for controlling an individually customized sleep massage chair using sleep state data, and more particularly to a system and a method for controlling an individually customized sleep massage chair using sleep state data, which induces a user to enter the sleep state, analyzes the sleep state of the user, thereby providing a result thereof, and performs control so as to change the shape of the chair according to the analysis result such that the user remains in the sleep state.

### 2. Description of the Prior Art

Recently, a variety of fitness devices have been developed and widely used in real life in order to maintain or improve health and physical strength or in order to recover from fatigue and relieve stress at homes and in health clubs.

In particular, devices capable of giving a massage are drawing attention because there is a growing demand for relaxing tight muscles and relieving fatigue and stress by means of a massage.

Massage is a supplementary medical-therapy that promotes blood circulation and relieves fatigue by sweeping, squeezing, pressing, pulling, tapping, or moving a body with the hands or special devices. Apparatuses or devices for performing a mechanical massage have been proposed.

Among the devices for performing a massage, a massage chair configured to give a massage is gradually spreading. The massage chair includes a seat, a backrest, an armrest, a leg massage part, and the like in order for a user to sit comfortably thereon and receive a massage, and each of the above components is equipped with a massage unit configured as a massage module for giving a massage while moving along the user's spine, hips, thighs, calves, arms, etc. or an air bag for massaging the body of the user by repeatedly inflating and deflating according to input and output of air. Recently, in order to take a comfortable sleep on the massage chair at the same time as or separately from the massage (e.g., whole body massage), Korean Patent No. 10-1179019 entitled “Massage chair for improving sleep” and Korean Patent No. 10-1288406 entitled “Massage chair with deep sleeping inducer, and the like disclose massage chairs that allow the user to take a comfortable sleep while receiving a massage on the massage chairs.

However, although the conventional massage chairs having sleep induction and sleep control functions described above induce the user to enter the sleep state, the massage chairs cannot check whether or not the user has entered the sleep state, and cannot provide a function of providing a proper sleeping posture to the user and maintaining the same.

### SUMMARY OF THE INVENTION

In order to solve the problems above, an aspect of the present disclosure provides a system and a method for controlling an individually customized sleep massage chair using sleep state data, which induces a user to enter the sleep state, analyzes the sleep state of the user, thereby providing a result thereof, and performs control so as to change the shape of the chair according to the analysis result such that the user remains in the sleep state.

In view of the above aspect, the present disclosure includes: a massage chair configured to perform a massage on the body of a user, detect sitting posture data and weight data of the user, thereby transmitting the same to a terminal, and perform a modification operation of changing the posture of the user or a massage according to an operation control signal transmitted from the terminal; a terminal configured to, when a signal for the sleep mode is input by the user, receive the posture data and weight data measured by the massage chair and biometric data of the user measured by a wearable sensor part, calculate a relaxation index of the user using a pre-stored analysis program, output an operation control signal according to the calculated relaxation index such that the shape of the massage chair is changed, thereby inducing the user to enter a sleep state, compare the calculated relaxation index with a predetermined sleep determination reference value, if the relaxation index is equal to or greater than the reference value, determine that the user is in the sleep state, and perform control so as to maintain the sleep state of the user through operation control of the massage chair using the weight data of the user on the massage chair and adjustment of horizontality of the massage chair; and a wearable sensor part configured to measure the biometric data while being worn on the body of the user and transmit the measured biometric data to the terminal.

In addition, the present disclosure further includes a camera provided in the massage chair so as to photograph a face of the user.

In addition, the massage chair according to the present disclosure includes: a massage module including a seat, a backrest, an armrest, a leg rest, and a headrest supporting the hips, back, arms, legs, and head of the user, respectively, wherein the seat, the backrest, the armrest, the leg rest, and the headrest are rotatably connected and force is applied to at least one of the seat, the backrest, the armrest, and the leg rest, thereby performing a massage on the body of the user; a wireless receiver configured to receive the operation control signal transmitted from the terminal; position sensors provided in the massage module so as to measure the posture data and weight data of the user according to a change in the shape of the massage module; a massage chair controller configured to output a driving signal for control of changing the positions of the seat, the backrest, the armrest, the leg rest, and the headrest of the massage module and a massage signal according to the received operation control signal, and configured to perform control such that the measured posture data and weight data are transmitted to the terminal; a wireless transmitter configured to transmit the posture data and weight data to the terminal; and a massage module driving part configured to provide driving force to operate the massage module according to the driving signal and the massage signal.

In addition, the terminal according to the present disclosure is configured to receive facial image data photographed by the camera, compare a facial image of the user with a predetermined facial expression image, and reflect, to the relaxation index, a parameter value predetermined according to a selected comparative facial expression.

In addition, the terminal according to the present disclosure includes: a wireless receiver configured to receive posture data, weight data, biometric data, and photographic data transmitted from the massage chair, the wearable sensor part, and the camera; a data analyzer configured to receive posture data and weight data measured by the massage chair, biometric data of the user measured by the wearable sensor part, and facial image data of the user photographed by the camera, calculate a relaxation index of the user using an analysis program, output an operation control signal according to the calculated relaxation index so as to change the shape of the massage chair, thereby inducing the user to enter the sleep state, compare the calculated relaxation index with a predetermined sleep determination reference value, and if it is determined that the user is in the sleep state, perform operation control of the massage chair using the weight data of the user on the massage chair and adjustment of the horizontality of the massage chair, thereby maintaining the sleep state of the user; a wireless transmitter configured to transmit the operation control signal to the massage chair; and a display configured to visualize and display sleep entry time data and average sleeping time data of the user in a predetermined output format.

In addition, the data analyzer is configured to analyze stress data and fatigue data using the biometric data of the user and is configured to perform control so as to visualize and display a result of the analysis.

In addition, the wearable sensor part includes: a biometric data measuring part configured to be worn on the body of the user so as to measure a biometric signal; a sensor controller configured to output biometric data by eliminating a noise signal from the measured biometric signal; and a wireless transmitter configured to transmit the biometric data to the terminal.

In addition, the biometric signal includes at least one of a pulse, a body temperature, a heart rate, and oxygen saturation of the blood.

In addition, the present disclosure includes the steps of: (a) letting a terminal receive posture data and weight data of a user measured by a massage chair and biometric data of the user measured by a wearable sensor part when a sleep mode setting signal is input by the user; (b) letting the terminal calculate a relaxation index of the user by analyzing the received data in the step (a) using an analysis program and output an operation control signal according to the calculated relaxation index such that the shape of the massage chair is changed, thereby inducing the user to enter the sleep state; (c) letting the terminal compare the calculated relaxation index with a predetermined sleep determination reference value, thereby determining whether or not the user is in the sleep state; (d) if the user is in the sleep state as a result of the determination, letting the terminal receive biometric data of the user measured by the wearable sensor part, weight data of the user on the massage chair, and horizontality data of the massage chair, update the relaxation index, and control the operation of the massage chair so as to maintain the sleep state; and (e) when the sleep state of the user is terminated, letting the terminal store data analyzed while asleep, convert the analyzed data to visualized data in a predetermined output format, and output the same.

In addition, the step (b) according to the present disclosure further includes: receiving a facial image of the user photographed by a camera; comparing the received facial image of the user with a predetermined facial expression image; and reflecting a parameter value predetermined according to a selected comparative facial expression to the relaxation index.

Furthermore, the step (d) according to the present disclosure further includes: analyzing stress data and fatigue data of the user from the biometric data of the user; normalizing the analyzed stress data and fatigue data; and outputting the stress index and the fatigue index.

The present disclosure can provide individually customized sleeping functions to the user by analyzing facial data, biometric data, and posture data of the user, by inducing the user to enter the sleep state, and by controlling the posture and position of the chair on the basis of the analyzed data such that the user remains in the sleep state.

In addition, the present disclosure can provide the sleep entry time, the average sleeping time, and the user's health data on the basis of sleep data of the user.

### BRIEF DESCRIPTION OF THE DRAWINGS

The above and other aspects, features, and advantages of the present disclosure will be more apparent from the following detailed description taken in conjunction with the accompanying drawings, in which:

FIG. 1 is a perspective view illustrating a system for controlling an individually customized sleep massage chair using sleep state data according to the present disclosure;

FIG. 2 is a block diagram illustrating the configuration of a massage chair of a system for controlling an individually customized sleep massage chair using sleep state data according to the present disclosure;

FIG. 3 is a block diagram illustrating the configuration of a terminal in a system for controlling an individually customized sleep massage chair using sleep state data according to the present disclosure;

FIG. 4 is a block diagram illustrating the configuration of a sensor in a system for controlling an individually customized sleep massage chair using sleep state data according to the present disclosure; and

FIG. 5 is a flowchart illustrating a control procedure of a system for controlling an individually customized sleep massage chair using sleep state data according to the present disclosure.

### DETAILED DESCRIPTION OF THE EXEMPLARY EMBODIMENTS

Hereinafter, a preferred embodiment of a system and a method for controlling an individually customized sleep massage chair using sleep state data according to the present disclosure will be described in detail with reference to the accompanying drawings.

In the present specification, the expression “a part includes an element” does not mean that the part excludes other elements, but means that the part may further include other elements.

In addition, the term “parr, “-er (or)”, “module, or the like means a unit for processing at least one function or operation, and may be implemented as hardware, software, or a combination thereof.

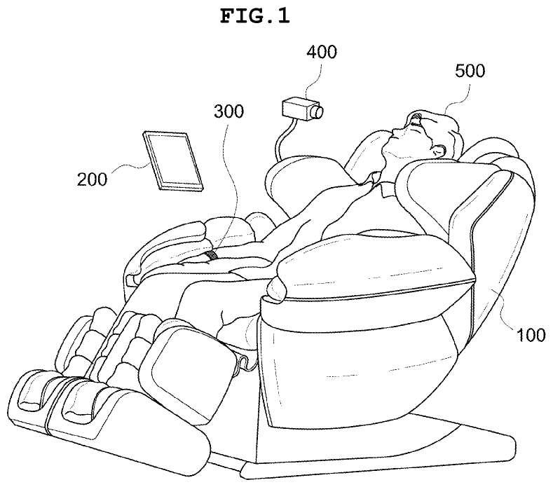

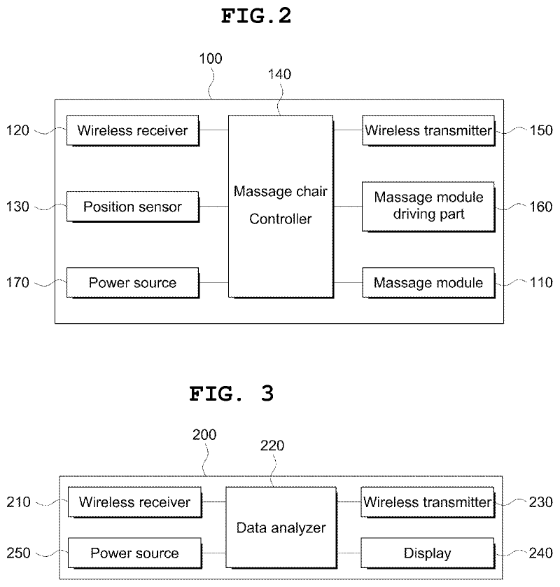

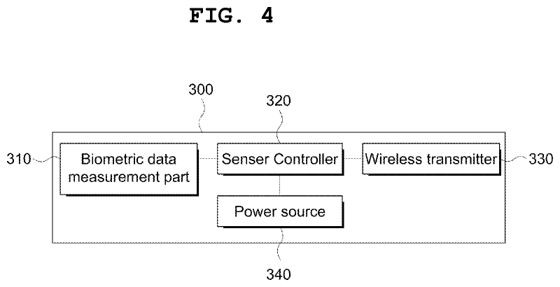

FIG. 1 is a perspective view illustrating a system for controlling an individually customized sleep massage chair using sleep state data according to the present disclosure; FIG. 2 is a block diagram illustrating the configuration of a massage chair of a system for controlling an individually customized sleep massage chair using sleep state data according to the present disclosure; FIG. 3 is a block diagram illustrating the configuration of a terminal in a system for controlling an individually customized sleep massage chair using sleep state data according to the present disclosure; and FIG. 4 is a block diagram illustrating the configuration of a sensor in a system for controlling an individually customized sleep massage chair using sleep state data according to the present disclosure.

As shown in FIGS. 1 to 4, a system for controlling an individually customized sleep massage chair according to the present disclosure is configured to include a massage chair 100 providing a sleep mode and a massage mode, a terminal 200 for controlling the operation of the massage chair 100, a wearable sensor part 300 for measuring biometric data of the user 500, and a camera 400 for photographing a user's face.

The massage chair 100 is configured to perform a massage on the body of a user 500, detect sitting posture data and weight data of the user 500, thereby transmitting the same to the terminal 200, and perform a modification operation of changing the posture of the user 500 or perform a massage according to an operation control signal transmitted from the terminal 200. The massage chair 100 is configured to include a massage module 110, a wireless receiver 120, position sensors 130, a massage chair controller 140, a wireless transmitter 150, a massage module driving part 160, and a power source 170.

The massage module 110 includes a seat, a backrest, an armrest, a leg rest, and a headrest supporting the hips, back, arms, legs, and head of the user, respectively, and the seat, the backrest, the armrest, the leg rest, and the headrest are rotatably connected. Force is applied to at least one of the seat, the backrest, the armrest, and the leg rest, thereby performing a massage operation on the body of the user.

The wireless receiver 120 is configured to receive an operation control signal transmitted from the terminal 200 using Bluetooth, ZigBee, RF communication, or the like.

A plurality of position sensors 130 are provided in the massage module 110 and are configured to measure posture data of the user 500 and weight data of the user 500 according to a change in the shape of the massage module 110. The position sensors 130 are provided in the seat, the backrest, the armrest, the leg rest, and the headrest, respectively. When the hips, back, arms, legs, head, etc. of the user 500 come into contact with, or are placed on, the seat, the backrest, the armrest, the leg rest, and the headrest at the respective positions, the position sensors 130 measure the positions and weights thereof, thereby obtaining posture data of the user 500 and weight data of the user 500 distributed on the massage module 110.

The position sensor 130 may be a pressure sensor for detecting pressure in order to simultaneously obtain positions and weights, but it is not limited thereto, and any detection means capable of simultaneously measuring positions and weights can be employed as the position sensor 130.

When an operation control signal is received from the terminal 200, the massage chair controller 140 outputs a driving signal for control such that the positions of the seat, the backrest, the armrest, the leg rest, and the headrest of the massage module 110 are changed and a massage signal to the massage module driving part 160 according to the received operation control signal, and performs control such that the posture data and the weight data of the user, which are measured by the position sensors 130, are transmitted to the terminal 200.

The wireless transmitter 150 is configured to transmit the posture data and the weight data to the terminal 200 and receive an operation control signal using Bluetooth, ZigBee, RF communication, or the like.

The massage module driving part 160 is configured to provide driving force to operate the massage module 110 according to the driving signal and the massage signal output from the massage chair controller 140 and includes a plurality of operating means such as actuators, motors, and the like

That is, the massage module driving part 160 changes the positions and angles of arrangement of the seat, the backrest, the armrest, the leg rest, and the headrest of the massage module 110 according to the driving signal, thereby adjusting the horizontality thereof or realizing a comfortable posture (e.g., a weightless posture) of the user 500 so as to minimize the effect of the gravity applied to the body of the user 500.

The power source 170 is configured to supply power necessary for the operation of the massage chair 100 so that the massage chair 100 operates using commercial power.

The terminal 200 is configured to receive operation selection data, such as a sleep mode or a massage mode, from the user 500 and perform control such that the massage chair 100 operates in the sleep mode or the massage mode according to the received operation selection data. The terminal 200 is configured to include a wireless receiver 210, a data analyzer 220, a wireless transmitter 230, a display 240, and a power source 250.

In addition, the terminal 200 may be configured as a mobile terminal, in which application programs can be installed, using wireless communication, but it is not limited thereto, and may be configured so as to be connected to the massage chair 100 using cables or the like.

In addition, when a signal for the sleep mode is input, the terminal 200 receives posture data and weight data measured by the massage chair 100 and biometric data of the user 500 measured by the wearable sensor part 300 and calculates a relaxation index of the user using a pre-stored analysis program.

In addition, the terminal 200 outputs an operation control signal according to the calculated relaxation index such that the shape of the massage chair 100 is changed, thereby inducing the user 500 to enter the sleep state.

In addition, the terminal 200 compares the calculated relaxation index with a predetermined sleep determination reference value. If the relaxation index is equal to or greater than the reference value, the terminal 200 determines that the user 500 is in the sleep state and performs control so as to maintain the sleep state of the user 500 through operation control using the weight data of the user 500 on the massage chair 100 and adjustment of the horizontality of the massage chair 100.

In addition, the terminal 200 receives facial image data of the user 500, which is photographed by a camera 400, and compares a facial image of the user 500 with a predetermined facial expression image, thereby reflecting, in the relaxation index, a parameter value set to a similar comparative facial expression selected within a specific range.

The wireless receiver 210 is configured to receive posture data, weight data, biometric data, and photographic data transmitted from the massage chair 100, the wearable sensor part 300, and the camera 400 using Bluetooth, ZigBee, RF communication, or the like.

When a signal for the sleep mode is input by the user 500, the data analyzer 220 outputs a start signal to the massage chair 100, the wearable sensor part 300, and the camera 400 and receives posture data and weight data measured by the massage chair 100, biometric data of the user 500 measured by the wearable sensor part 300, and facial image data of the user 500 photographed by the camera 400.

In addition, the data analyzer 220 calculates a relaxation index of the user using a program of analyzing the received posture data, weight data, biometric data, and facial image data.

The relaxation index is calculated by analyzing and normalizing the posture of the user 500 depending on the contact state with the massage chair 100, the stable state of the user using the user's pulse and heart rate, the facial expression of the user, and the like.

In addition, the data analyzer 220 outputs an operation control signal according to the calculated relaxation index such that the massage chair 100 (i.e., the massage module 110) has a horizontal position, a weightless position, or the like, thereby inducing the user 500 to enter the sleep state.

In addition, the data analyzer 220 continuously updates the calculated relaxation index after performing the sleep entry induction, compares the updated relax index with a predetermined sleep determination reference value, and, if it is determined that the user 500 is in the sleep state, outputs an operation control signal for maintaining the sleep state of the user 500 so as to perform operation control using angle control, such as adjustment of the horizontality of the massage chair 100 or the like, by taking into account a lying posture (i.e., a supine posture, a recumbent posture, or the like) of the user 500 through the weight data of the user 500 distributed on the massage module 110 of the massage chair 100.

In addition, the data analyzer 220 calculates time data taken for the user 500 to enter the sleep state and average sleeping time data after the entry into the sleep state and provides visualized data obtained by converting the calculated sleep entry time data and average sleeping time data of the user 500 into a predetermined output format.

In addition, the data analyzer 220 analyzes stress data and fatigue data of the user 500 using the biometric data measured by the wearable sensor part 300 while asleep and displays data visualized by converting the analysis result into a predetermined format.

That is, the data analyzer 220 calculates a stress index by normalizing the pulse, the body temperature, the heart rate, etc. of the user 500 included in the biometric data into the stress data using an analysis program and calculates a fatigue index by normalizing the oxygen saturation data included in the blood of the user 500 into the fatigue data using an analysis program, thereby providing the same.

In addition, the data analyzer 220 may output, to the massage chair 100, an operation control signal on the basis of the analyzed relaxation index, stress index, and fatigue index so as to perform control such that the sleeping posture of the user 500 is maintained or changed, or so as to provide a proper massage function.

Furthermore, the data analyzer 220 may analyze male/female/young/old data using facial image data of the user photographed by the camera 400, and may output, to the massage chair 100, an operation control signal on the basis of the analyzed data so as to perform control such that the sleeping posture of the user is maintained or changed, or so as to provide a proper massage function.

The wireless transmitter 230 is configured to transmit an operation control signal to the massage chair 100 and the wearable sensor part 300 using Bluetooth, ZigBee, RF communication, or the like.

The display 240 is configured to output visualized data output from the data analyzer 220 and is configured to visualize and display the sleep entry time data, the average sleeping time data, the stress index, the fatigue index, etc. of the user 500 in a predetermined output format. The power source 250 is configured to supply operating power to the terminal 200 and includes a battery embodied as a primary battery or a secondary battery.

The wearable sensor part 300 is configured to measure biometric data while being worn on the body of the user 500 and transmit the measured biometric data to the terminal 200, and is configured to include a biometric data measurement part 310, a sensor controller 320, a wireless transmitter 330, and a power source 340.

The biometric data measurement unit 310 measures a biometric signal while being worn on the body of the user 500 (i.e., on the wrist of the user).

In addition, the biometric data measurement part 310 measures a pulse, which is a wave signal generated by blood in the heart, body temperature, heart rate, and the oxygen saturation of blood, wherein the oxygen saturation may be obtained as a signal using a linear relationship between the blood volume, which changes with the heart contraction and relaxation, and the amount of light absorbed into the hemoglobin in the blood.

The sensor controller 320 removes a noise signal from the biometric signal measured by the biometric data measurement unit 310 and outputs biometric data obtained by converting the biometric signal from which the noise signal has been removed into a digital signal.

The wireless transmitter 330 is configured to transmit the biometric data to the terminal 200 and receive an operation control signal using Bluetooth, ZigBee, RF communication, or the like.

The power source 340 is configured to supply operating power to the wearable sensor part 300 and includes a battery embodied as a primary battery or a secondary battery.

The camera 400 is configured to output facial image data obtained by photographing a face of the user 500, is provided in the massage chair 100, and includes a camera using a CDD sensor, a CMOS sensor, or the like. However, the camera 400 is not limited to the above description, and any photoelectric transformation means for converting an optical signal into an electric signal may be employed as the camera 400.

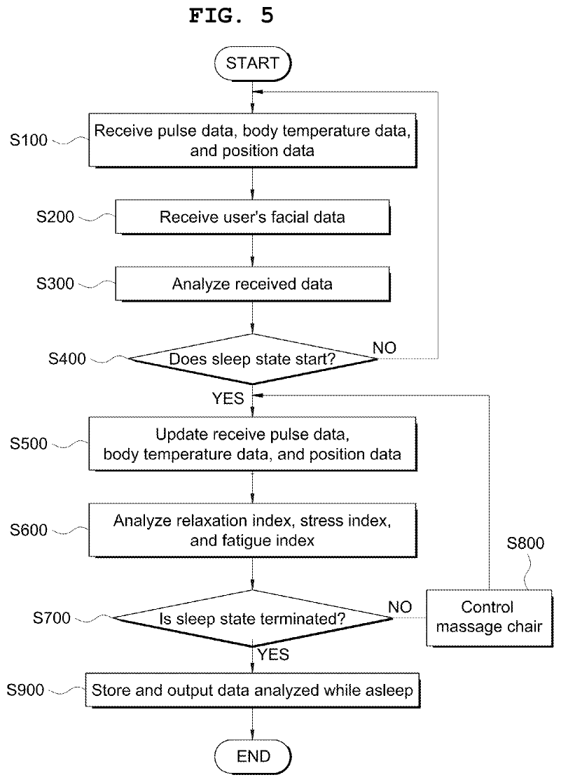

Next, a method for controlling an individually customized sleep massage chair using sleep state data, according to the present disclosure, will be described with reference to FIGS. 1 to 5.

When a sleep-mode setting signal is received from the user 500, the terminal 200 receives posture data and weight data of the user 500 measured by the massage chair 100 and biometric data of the user 500 measured from the wearable sensor part 300 (S100).

In addition, the terminal 200 may further receive facial image data of the user 500 photographed by the camera 400 (S200).

The terminal 200 analyzes the information received in steps S100 and S200 using an analysis program, thereby calculating a relaxation index of the user 500, and outputs an operation control signal according to the calculated relaxation index so as to change the shape of the massage chair 100, thereby inducing the user 500 to enter the sleep state (S300).

Further, in step S300, the terminal 200 compares the facial image of the user 500 photographed by the camera 400 with a predetermined facial expression image and reflects a predetermined parameter value according to a selected comparative facial expression to the relaxation index.

After performing step S300, the terminal 200 compares the calculated relaxation index with a predetermined sleep determination reference value, thereby determining whether or not the user 500 is in the sleep state (S400).

If the user 500 is in the sleep state as a result of the determination in step S400, the terminal 200 receives the biometric data of the user 500 measured by the wearable sensor part 300, the weight data of the user 500 on the massage chair 100, and the horizontality data of the massage chair 100, thereby updating the relaxation index (S500), and controls the operation of the massage chair 100 so as to maintain the sleep state of the user 500 (S800).

In addition, in step S500, the terminal 200 further performs a process of analyzing stress data and fatigue data of the user 500 from the biometric data of the user 500 (S600) and outputs a stress index and a fatigue index by normalizing the analyzed stress data and fatigue data.

Meanwhile, when the sleep state of the user 500 is terminated (S700), the terminal 200 stores time data taken for the user 500 to enter the sleep state, average sleeping time data after the entry into the sleep state, the stress data, and the fatigue data and outputs data visualized by converting the data into a predetermined format (S900).

Therefore, the present disclosure can provide a customized sleeping function by analyzing the facial data, the biometric data, and the posture data of the user to thus induce the user to enter the sleep state and by controlling the posture and position of the chair on the basis of the analyzed data such that the user remains in the sleep state.

While the present disclosure has been described with reference to preferable embodiments, it is to be understood that the present disclosure may be modified and changed in various forms by those skilled in the art without departing from the scope and spirit of the present disclosure as defined in the appended claims.

In addition, the reference numerals shown in the claims are merely intended for clarity and convenience of explanation, and the present disclosure is not limited thereto. The thicknesses of the lines or the sizes of the elements illustrated in the drawings of the embodiments may be exaggerated for clarity and convenience of explanation. The terms used above have been defined in consideration of the functions thereof in the present disclosure, but may be varied according to the intention or practices of a user or operator, so that the interpretation of the terms must be made on the basis of the entire content of this specification.

What is claimed is:

1. A system for controlling an individually customized sleep massage chair using sleep state data, the system comprising:
a massage chair (100) configured to perform a massage on a body of a user (500), detect sitting posture data and weight data of the user (500), thereby transmitting the same to a terminal (200), and perform a modification operation of changing a posture of the user (500) or a massage according to an operation control signal transmitted from the terminal (200);
a terminal (200) configured to, when a signal for a sleep mode is input by the user (500), receive the posture data and the weight data measured by the massage chair (100) and biometric data of the user (500) measured by a wearable sensor part (300), calculate a relaxation index of the user using a pre-stored analysis program, output an operation control signal according to the calculated relaxation index such that a shape of the massage chair (100) is changed, thereby inducing the user (500) to enter a sleep state, compare the calculated relaxation index with a predetermined sleep determination reference value, if the relaxation index is equal to or greater than the reference value, determine that the user (500) is in the sleep state, and perform control so as to maintain the sleep state of the user (500) through operation control of the massage chair (100) using the weight data of the user (500) on the massage chair (100) and adjustment of horizontality of the massage chair (100); and
a wearable sensor part (300) configured to measure the biometric data while being worn on the body of the user (500) and transmit the measured biometric data to the terminal (200).

2. The system of claim 1, further comprising a camera (400) provided in the massage chair (100) so as to photograph a face of the user (500).

3. The system of claim 2, wherein the massage chair 100 comprises:
a massage module (110) comprising a seat, a backrest, an armrest, a leg rest, and a headrest supporting the hips, back, arms, legs, and head of the user, respectively, wherein the seat, the backrest, the armrest, the leg rest, and the headrest are rotatably connected and force is applied to at least one of the seat, the backrest, the armrest, and the leg rest, thereby performing a massage on the body of the user;
a wireless receiver (120) configured to receive the operation control signal transmitted from the terminal (200);
position sensors (130) provided in the massage module (110) so as to measure the posture data and weight data of the user according to a change in the shape of the massage module (110);
a massage chair controller (140) configured to output a driving signal for performing control of changing the positions of the seat, the backrest, the armrest, the leg rest, and the headrest of the massage module (110) and a massage signal according to the received operation control signal, and configured to perform control such that the measured posture data and weight data are transmitted to the terminal (200);
a wireless transmitter (150) configured to transmit the posture data and weight data to the terminal (200); and
a massage module driving part (160) configured to provide driving force to operate the massage module (110) according to the driving signal and the massage signal.

4. The system of claim 2, wherein the terminal (200) is configured to receive facial image data photographed by the camera (400), compare a facial image of the user (500) with a predetermined facial expression image, and reflect, in the relaxation index, a parameter value predetermined according to a selected comparative facial expression.

5. The system of claim 4, wherein the terminal 200 comprises:
a wireless receiver (210) configured to receive posture data, weight data, biometric data, and photographic data transmitted from the massage chair (100), the wearable sensor part (300), and the camera (400);
a data analyzer (220) configured to receive posture data and weight data measured by the massage chair (100), biometric data of the user (500) measured by the wearable sensor part (300), and facial image data of the user (500) photographed by the camera (400), calculate a relaxation index of the user using an analysis program, output an operation control signal according to the calculated relaxation index so as to change the shape of the massage chair (100), thereby inducing the user (500) to enter a sleep state, compare the calculated relaxation index with a predetermined sleep determination reference value, and if it is determined that the user (500) is in the sleep state, perform operation control of the massage chair (100) using the weight data of the user (500) on the massage chair (100) and adjustment of the horizontality of the massage chair (100), thereby maintaining the sleep state of the user (500);
a wireless transmitter (230) configured to transmit the operation control signal to the massage chair (100); and
a display (240) configured to visualize and display sleep entry time data and average sleeping time data of the user (500) according to a predetermined output format.

6. The system of claim 5, wherein the data analyzer (220) is configured to analyze stress data and fatigue data using the biometric data of the user (500) and is configured to perform control so as to visualize and display a result of the analysis.

7. The system of claim 2, wherein the wearable sensor part (300) comprises:
a biometric data measuring part (310) configured to be worn on the body of the user (500) so as to measure a biometric signal;
a sensor controller (320) configured to output biometric data by eliminating a noise signal from the measured biometric signal; and
a wireless transmitter (330) configured to transmit the biometric data to the terminal (200).

8. The system of claim 7, wherein the biometric signal comprises at least one of a pulse, a body temperature, a heart rate, and oxygen saturation of the blood.

9. A method for controlling an individually customized sleep massage chair, the method comprising the steps of:
(a) letting a terminal (200) receive posture data and weight data of a user (500) measured by a massage chair (100) and biometric data of the user (500) measured by a wearable sensor part (300) when a sleep mode setting signal is input by the user (500);
(b) letting the terminal (200) calculate a relaxation index of the user by analyzing the received data in the step (a) using an analysis program and output an operation control signal according to the calculated relaxation index such that a shape of the massage chair (100) is changed, thereby inducing the user (500) to enter a sleep state;
(c) letting the terminal (200) compare the calculated relaxation index with a predetermined sleep determination reference value, thereby determining whether or not the user (500) is in the sleep state;
(d) if the user is in the sleep state as a result of the determination, letting the terminal (200) receive biometric data of the user (500) measured by the wearable sensor part (300), weight data of the user (500) on the massage chair (100), and horizontality data of the massage chair (100), update the relaxation index, and control the operation of the massage chair (100) so as to maintain the sleep state; and
(e) when the sleep state of the user (500) is terminated, letting the terminal (200) store data analyzed during asleep, convert the analyzed data to visualized data in a predetermined output format, and output the same.

10. The system of claim 9, wherein the step (b) further comprises:
receiving a facial image of the user (500) photographed by a camera (400);
comparing the received facial image of the user (500) with a predetermined facial expression image; and
reflecting a parameter value predetermined according to a selected comparative facial expression to the relaxation index.

11. The system of claim 9, wherein the step (d) further comprises:
analyzing stress data and fatigue data of the user 500 from the biometric data of the user 500;
normalizing the analyzed stress data and fatigue data; and
outputting the stress index and the fatigue index.
